(19) United States
(12) Patent Application Publication
(10) Pub. No.: US 2006/0013788 A1
(43) Pub. Date: Jan. 19, 2006

(54) Cosmetic composition for caring for or making up keratin materials, comprising at least one polyester, and method of use thereof

(76) Inventor: Vanina Filippi, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/179,463

(22) Filed: Jul. 13, 2005

Related U.S. Application Data
(60) Provisional application No. 60/591,625, filed on Jul. 28, 2004.

(30) Foreign Application Priority Data
Jul. 13, 2004 (FR) 04 51514

(51) Int. Cl. A61K 8/85

(52) U.S. Cl. 424/70.11

(57) ABSTRACT

The present disclosure relates to a cosmetic composition, for caring for or making up keratin materials, comprising, in a cosmetically acceptable medium, i) at least one polyester derived from the esterification, with a polycarboxylic acid, of an aliphatic hydroxycarboxylic acid ester comprising at least two hydroxyl groups and ii) at least one volatile oil. The present disclosure also relates to a method of imparting to a cosmetic composition at least one cosmetic property chosen from gloss, staying-power and comfort properties.

This application claims benefit of U.S. Provisional Application No. 60/591,625, filed Jul. 28, 2004, the contents of which are incorporated herein by reference. This application also claims benefit of priority under 35 U.S.C. § 119 to French patent application No. 04 51514, filed Jul. 13, 2004, the contents of which are also incorporated herein by reference.

The present disclosure relates to a cosmetic composition for making up or caring for the skin, of either the human face or body, and the lips and/or the integuments, for instance the hair, the eyelashes, the eyebrows or the nails, comprising a cosmetically acceptable medium comprising at least one particular polyester and at least one volatile oil. The compositions as disclosed herein can have noteworthy cosmetic properties, and can, for example, give the makeup or care treatment gloss, staying-power and comfort properties simultaneously.

The compositions of the present disclosure may, for instance, be in the form of lipstick or a lip gloss, a makeup rouge, an eyeshadow, a tattoo product, a mascara, an eyeliner, a nail varnish, an artificial tanning product for the skin, a hair coloring product, or a haircare product.

The present inventor has found, surprisingly, that the addition, in a cosmetic composition, of i) at least one polyester derived from the esterification, with a polycarboxylic acid, of an aliphatic hydroxycarboxylic acid ester, wherein the ester comprises at least two hydroxyl groups, and ii) at least one volatile oil, can make it possible to obtain a composition that can be glossy and have good staying-power, that can also have good comfort and/or migration-resistance properties.

Thus, the present disclosure relates to a cosmetic composition, for example, for caring for or making up keratin materials, comprising, in a cosmetically acceptable medium, i) at least one polyester derived from the esterification, with a polycarboxylic acid, of an aliphatic hydroxycarboxylic acid ester, wherein the ester comprises at least two hydroxyl groups and ii) at least one volatile oil.

The present disclosure also relates to a cosmetic process for giving to a film of a cosmetic composition at least one property chosen from gloss, staying-power and comfort properties, comprising introducing into the composition i) at least one polyester derived from the esterification, with a polycarboxylic acid, of an aliphatic hydroxycarboxylic acid ester, wherein the ester comprises at least two hydroxyl groups and ii) at least one volatile oil.

The present disclosure further relates to the use of the combination of i) at least one polyester derived from the esterification, with a polycarboxylic acid, of an aliphatic hydroxycarboxylic acid ester comprising at least two hydroxyl groups and ii) at least one volatile oil, to obtain a cosmetic composition endowed with at least one property chosen from gloss, staying-power and comfort properties.

The present disclosure still further relates to the use of i) at least one polyester derived from the esterification, with a polycarboxylic acid, of an aliphatic hydroxycarboxylic acid ester comprising at least two hydroxyl groups and ii) at least one volatile oil, in a physiologically acceptable composition, as agents for giving the composition at least one property chosen from gloss, staying-power and comfort properties.

The At Least One Polyester

The at least one polyester according to the present disclosure derives from the esterification

of a polycarboxylic acid, and

of an aliphatic hydroxycarboxylic acid ester (referred to hereinbelow as “hydroxylated ester”). The aliphatic hydroxycarboxylic acid ester (or hydroxylated ester) comprises at least two hydroxyl groups.

For example, the hydroxylated ester can be derived from the reaction of at least one hydroxylated aliphatic carboxylic acid with at least one polyol.

The at least one hydroxylated aliphatic acid can comprise, for instance, from 2 to 40 carbon atoms, for example from 10 to 34 carbon atoms, such as from 12 to 28 carbon atoms; it can also comprise from 1 to 20 hydroxyl groups, for instance from 1 to 10 hydroxyl groups, such as from 1 to 6 hydroxyl groups, which may be subsequently esterified with the polycarboxylic acid to obtain the polyester of the present disclosure.

The at least one polyol may comprise from 2 to 40 carbon atoms, such as from 3 to 30 carbon atoms. The at least one polyol can be, for example, an aliphatic polyol. In one embodiment of the present disclosure, the at least one polyol is not a saccharide.

The at least one polyol that reacts with the hydroxylated acid described above may be partially or totally esterified; for example, in at least one embodiment of the present disclosure, the polyol is totally esterified.

The at least one aliphatic hydroxycarboxylic acid ester, for instance, can be a hydroxylated fatty acid ester such that the fatty acid residue comprises at least 12 carbon atoms, for example from 12 to 40 carbon atoms, such as from 12 to 28 carbon atoms.

The at least one aliphatic hydroxycarboxylic acid ester that may be used according to the present disclosure can be chosen from:
a) partial or total esters of saturated linear monohydroxylated aliphatic monocarboxylic acids;
b) partial or total esters of unsaturated monohydroxylated aliphatic monocarboxylic acids such as glyceryl triricinoleate (castor oil); and
c) partial or total esters of a C₂ to C₁₆ aliphatic polyol that has reacted with a monohydroxylated or polyhydroxylated aliphatic monocarboxylic or polycarboxylic acid, for instance, such as triglycerides, esters of pentaerythritol, of trimethylolpropane, of propylene glycol, of neopentyl glycol, of dipentaerythritol or of polyglycerol, and sorbitol esters; and mixtures thereof.

For example, in at least one embodiment of the present disclosure, when the at least one aliphatic hydroxycarboxylic acid ester results from the esterification of an aliphatic polycarboxylic acid such as those mentioned above, there remain no residue COOH groups that are not engaged in an ester bond.

The at least one aliphatic hydroxycarboxylic acid ester can be chosen from, for instance, esters of C₂ to C₁₆ aliphatic polyols, wherein the polyols have reacted with a hydroxylated aliphatic fatty acid with a saturated or unsaturated chain, comprising at least 12 carbon atoms. In at least one embodiment of the present disclosure, the at least one fatty acid is ricinoleic acid and the aliphatic hydroxycarboxylic acid ester is hydrogenated castor oil.

Hydrogenated castor oil is commercially available. It is, for example, the product Himako P® from Hawaken Fine Chemicals.

The polycarboxylic acid comprises at least two COOH groups. In at least one embodiment of the present disclosure, for example, it is a diacid dimer of unsaturated aliphatic carboxylic acid(s).

The polycarboxylic acid according to the present disclosure can be, for instance, aliphatic, such as an aliphatic dicarboxylic acid.

According to one embodiment of the present disclosure, the polycarboxylic acid is a diacid dimer of unsaturated fatty acid(s), i.e., a dimer formed from at least one unsaturated fatty acid, for example from a single unsaturated fatty acid or from two different unsaturated fatty acids. The fatty acid can be, for example, monounsaturated or diunsaturated. As used herein, the term “fatty acid” is understood to mean an acid obtained by hydrolysis of a fatty substance of plant or animal origin.

The diacid dimers of unsaturated fatty acid(s), or alternatively diacid dimers, are conventionally obtained by an intermolecular dimerization reaction of at least one unsaturated fatty acid. For example, in at least one embodiment, only one type of unsaturated fatty acid is dimerized.

The diacid dimers of unsaturated fatty acid(s) can be obtained, for instance, by dimerization of an unsaturated fatty acid of C₈ to C₃₄, such as C₁₂ to C₂₂, for example, C₁₆ to C₂₀ and such as C₁₈.

Among the unsaturated fatty acids that may be mentioned, non-limiting examples include undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

In at least one embodiment of the present disclosure, for instance, the diacid dimer is obtained by dimerization of linoleic and/or linolenic acid. The commercial product Pripol 1009® from Uniqema can be used, for example.

The diacid dimer can, for example, be saturated, i.e. contain no carbon-carbon double bonds, and it can be obtained by condensation of unsaturated fatty acid(s) optionally followed by a hydrogenation, to convert any double bonds into single bonds.

The diacid dimers of unsaturated fatty acid(s) may also be obtained, for instance, by dimerization of linoleic acid, optionally followed by hydrogenation of the dimer thus obtained. The hydrogenated form may be partial or total and may correspond, for example, to the saturated form, which is more stable to oxidation.

Diacid dimers, such as dilinoleic diacids whose oxidation stability has been improved by hydrogenation of the double bonds remaining after the dimerization reaction are also commercially available.

In the present disclosure, any diacid dimer currently commercially available may be used.

The at least one polyester according to the present disclosure can have a molecular weight ranging from 2,000 to 8,000 g/mol, such as from 3,000 to 7,000 g/mol, measured according to the calibration method with polystyrene using gel permeation chromatography under the following conditions:

| | |
|---|---|
| Machine | TOSOH SC-8010 System |
| Column | Shodex KF-800D + KF-805L × 2 |
| Eluent | THF |
| Temperature | Isothermic 40° C. column |
| Flow rate | 1.0 ml/min |
| Concentration | About 0.2 weight/vol % |
| Amount injected | 100 μl |
| Solubility | Total dissolution |
| Detector | Differential refractometer |

For example, Risocast DL-L® has a number-average molecular mass ranging from 3,500 to 4,000 g/mol, and Risocast DA-H® has a number-average molecular mass ranging from 6,000 to 6,500 g/mol. These products are sold by the Japanese company Kokyu Alcohol Kogyo.

The molar ratio between the at least one polycarboxylic acid and the at least one hydroxylated ester used to prepare the polyester according to the present disclosure can range from 0.20 to 1, such as, from 0.20 to 0.40. For example, this ratio is equal to 0.75 for Risocast DA-H® and is equal to 0.5 for Risocast DA-L®.

Non-limiting mention may be made, for instance, among the polyesters that may be used in the composition according to the present disclosure, of:

the ester resulting from the esterification reaction of hydrogenated castor oil with dilinoleic acid in proportions of 2 to 1, and

the ester resulting from the esterification reaction of hydrogenated castor oil with isostearic acid in proportions of 4 to 3.

The at least one polyester may comprise at least one compound chosen from those of formulae (1) and (2) or a mixture thereof:

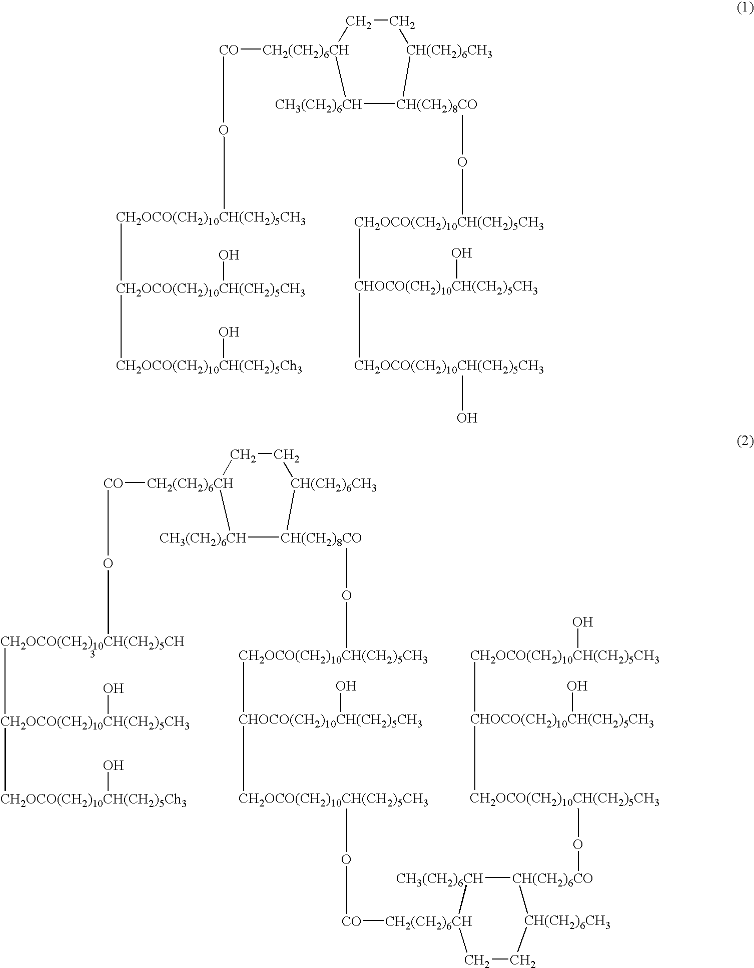

The at least one polyester of the present disclosure can be, for example, a compound that is pasty or viscous at room temperature (25° C.).

As used herein, the term “pasty” is understood to mean a polyester as described above, with a reversible solid/liquid change of state, and comprising at a temperature of 23° C. a liquid fraction and a solid fraction.

The polyester can have a hardness at 20° C., for example, ranging from 0.001 MPa to 0.5 MPa, such as from 0.002 MPa to 0.4 MPa.

The hardness is measured according to a method of penetration of a probe in a sample of compound and, for instance, using a texture analyzer (for example the TA-XT2i® machine from Rheo) equipped with a stainless-steel cylinder 2 mm in diameter. The hardness measurement is performed at 20° C. at the center of five samples. The cylinder is introduced into each sample at a pre-speed of 1 mm/s and then at a measuring speed of 0.1 mm/s, the penetration depth being 0.3 mm. The hardness value revealed is that of the maximum peak.

The at least one polyester may also, at a temperature of 23° C., be in the form of a liquid fraction and a solid fraction. In other words, the starting melting point of the polyester may be less than 23° C. The liquid fraction of the polyester measured at 23° C. can be present in an amount ranging from 9% to 97% by weight, relative to the weight of the compound. This liquid fraction at 23° C. can be present in an amount ranging from 15% to 85% by weight, such as ranging from 40% to 85% by weight.

The liquid fraction by weight of the polyester at 23° C. is equal to the ratio of the heat of fusion consumed at 23° C. to the heat of fusion of the polyester.

The heat of fusion of the polyester is the heat consumed by the compound to change from the solid state to the liquid state. The polyester is said to be in the solid state when all of its mass is in solid crystalline form. The polyester is said to be in the liquid state when all of its mass is in liquid form.

The heat of fusion of the polyester is equal to the area under the curve of the thermogram obtained using a differential scanning calorimeter (DSC), such as the calorimeter sold under the name MDSC 2920® by the company TA Instrument, with a temperature rise of 5° C. or 10° C. per minute, according to standard ISO 11357-3:1999. The heat of fusion of the polyester is the amount of energy required to make the compound change from the solid state to the liquid state. It is expressed in J/g.

The heat of fusion consumed at 23° C. is the amount of energy absorbed by the sample to change from the solid state to the state that it has at 23° C., comprising a liquid fraction and a solid fraction.

The liquid fraction of the polyester, measured at 32° C., can be present in an amount ranging from 30% to 100% by weight, relative to the weight of the compound, for instance from 80% to 100%, such as from 90% to 100% by weight, relative to the weight of the compound. When the liquid fraction of the polyester measured at 32° C. is equal to 100%, the temperature of the end of the melting range of the polyester is less than or equal to 32° C.

The liquid fraction of the polyester measured at 32° C. is equal to the ratio of the heat of fusion consumed at 32° C. to the heat of fusion of the polyester. The heat of fusion consumed at 32° C. is calculated in the same manner as the heat of fusion consumed at 23° C.

By way of non-limiting example, Risocast DA-L® has a hardness at 20° C. of 0.04 MPa, a liquid fraction at 23° C. equal to 82%, and a liquid fraction at 32° C. equal to 90%.

The at least one polyester of the composition according to the present disclosure may be present in an amount ranging from 1% to 99%, for example, from 1% to 75%, such as from 5% to 60%, and from 5% to 30%, for instance from 5% to 15% by weight, relative to the total weight of the composition.

The At Least One Volatile Oil

At least one volatile oil is included in the fatty phase of the composition.

As used herein, the term “volatile oil” is understood to mean an oil (or non-aqueous medium) capable of evaporating on contact with the skin in less than one hour, at room temperature and atmospheric pressure. The at least one volatile oil is a volatile cosmetic oil, which is liquid at room temperature, for example, having a non-zero vapor pressure, at room temperature and atmospheric pressure, for instance having a vapor pressure ranging from 0.13 Pa to 40,000 Pa (10⁻³ to 300 mmHg), such as ranging from 1.3 Pa to 13,000 Pa (0.01 to 100 mmHg), and ranging from 1.3 Pa to 1,300 Pa (0.1 to 10 mmHg).

In addition, the at least one volatile oil can have a boiling point, measured at atmospheric pressure, ranging from 150° C. to 260° C., such as ranging from 170° C. to 250° C.

As used herein, the term “hydrocarbon-based oil” is understood to mean an oil formed essentially from, or even comprising, carbon and hydrogen atoms, and possibly oxygen and nitrogen atoms, and comprising no silicon or fluorine atoms; it may comprise ester, ether, amine or amide groups.

As used herein, the term “silicone oil” is understood to mean an oil comprising at least one silicon atom, for instance, comprising Si—O groups.

As used herein, the term “fluoro oil” is understood to mean an oil containing at least one fluorine atom.

The at least one volatile oil may be, for example, a silicone oil or a hydrocarbon-based oil.

The volatile silicone oils that may be used in the present disclosure can be chosen from silicone oils with a flash point ranging from 40° C. to 102° C., for instance a flash point of greater than or equal to 55° C. and less than or equal to 95° C., such as ranging from 65° C. to 95° C.

Among the volatile silicone oils that may be used according to the present disclosure, non-limiting mention may be made of linear or cyclic silicones with a viscosity at room temperature of less than 8 cSt, for instance comprising from 2 to 7 silicon atoms, these silicones optionally comprising alkyl or alkoxy groups comprising from 1 to 10 carbon atoms. Among the volatile silicone oils that may be used according to the present disclosure, further non-limiting mention may be made of, for example, octamethyl-cyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Among the volatile silicone oils that may be used according to the present disclosure, non-limiting mention may be made of the silicones described in the French Patent Application No. FR 03/04259, now published as FR 2 853 227.

The at least one volatile hydrocarbon-based oil that may be used in the present disclosure may be chosen from hydrocarbon-based oils with a flash point ranging from 40° C. to 102° C., for instance ranging from 40° C. to 55° C., such as ranging from 40° C. to 50° C.

Volatile hydrocarbon-based oils that may be mentioned, by way of non-limiting example, include volatile hydrocarbon-based oils comprising from 8 to 16 carbon atoms and mixtures thereof, such as branched C₈-C₁₆ alkanes, for instance C₈-C₁₆ isoalkanes (also known as isoparaffins), isododecane, isodecane and isohexadecane and, for example, the oils sold under the trade names Isopar or Permetyl, branched C₈-C₁₆ esters, for instance isohexyl neopentanoate, and mixtures thereof. By way of further non-limiting example, the volatile hydrocarbon-based oil can be chosen from volatile hydrocarbon-based oils comprising from 8 to 16 carbon atoms, and mixtures thereof, such as from isododecane, isodecane and isohexadecane. In at least one embodiment, the at least one volatile hydrocarbon based oil is isododecane.

The at least one volatile oil can be present, for example, in an amount ranging from 5% to 97.5% of the total weight of the composition, for instance from 10% to 75% by weight, such as from 20% to 50% by weight, relative to the total weight of the composition.

The at least one volatile oil can be present, for instance, in an amount ranging from 20% to 50% by weight of the composition, such as from 30% to 40%, for example 35% by weight.

The at least one volatile oil can be present in an amount ranging from 40% to 60%, for instance from 45% to 55% by weight, relative to the weight of the liquid fatty phase.

Pasty Compound

The composition may also comprise at least one pasty compound in addition to the polyester described above.

As used herein, the term “pasty” is understood to mean a lipophilic fatty compound that undergoes a reversible solid/liquid change of state, having in the solid state an anisotropic crystal organization, and comprising at a temperature of 23° C. a liquid fraction and a solid fraction. The term “pasty” can also encompass polyvinyl laurate.

The pasty compound can be chosen from, for example:

lanolin and its derivatives,

polymer or non-polymer fluoro compounds,

polymer or non-polymer silicone compounds,

vinyl polymers, for instance:
olefin homopolymers,
olefin copolymers,
hydrogenated diene homopolymers and copolymers,
linear or branched oligomers, which are homopolymers or copolymers of alkyl (meth)acrylates for example, comprising a C₈-C₃₀ alkyl group,
oligomers, which are homopolymers and copolymers of vinyl esters comprising C₈-C₃₀ alkyl groups,
oligomers, which are homopolymers and copolymers of vinyl ethers comprising C₈-C₃₀ alkyl groups,
liposoluble polyethers resulting from the polyetherification between at least one C₂-C₁₀₀, such as C₂-C₅₀, diols,
esters,
   and mixtures thereof.

Among the liposoluble polyethers that may be used as disclosed herein, non-limiting mention may be made of copolymers of ethylene oxide and/or of propylene oxide with C₆-C₃₀ long-chain alkylene oxides, for example, such that the weight ratio of the ethylene oxide and/or of the propylene oxide to the alkylene oxides in the copolymer ranges from 5:95 to 70:30. In this family, non-limiting mention can further be made of copolymers such that the long-chain alkylene oxides are arranged in blocks with an average molecular weight ranging from 1,000 to 10,000, for example a polyoxyethylene/polydodecyl glycol block copolymer such as the ethers of dodecanediol (22 mol) and of polyethylene glycol (45 EO) sold under the brand name Elfacos ST9® by Akzo Nobel.

Among the pasty esters that may be used, non-limiting mention may be made of:
esters of a glycerol oligomer, for instance diglycerol esters, such as condensates of adipic acid and of glycerol, for which some of the hydroxyl groups of the glycerols have reacted with a mixture of fatty acids such as stearic acid, capric acid, stearic acid and isostearic acid and 12-hydroxystearic acid, for instance those sold under the brand name Softisan 649® by the company Sasol,
arachidyl propionate sold under the brand name Waxenol 801® by Akzo,
phytosterol esters,
non-crosslinked polyesters resulting from polycondensation between a linear or branched C₄-C₅₀ dicarboxylic acid or polycarboxylic acid and a C₂-C₅₀ diol or polyol, other than the polyester described above,
aliphatic esters of an ester resulting from the esterification of an aliphatic hydroxycarboxylic acid ester with an aliphatic monocarboxylic acid; and mixtures thereof, for instance
   the ester resulting from the esterification reaction of hydrogenated castor oil with isostearic acid in a ratio of 1 to 1 (1/1) or hydrogenated castor oil monoisostearate,
   the ester resulting from the esterification reaction of hydrogenated castor oil with isostearic acid in a ratio of 1 to 2 (1/2) or hydrogenated castor oil diisostearate,
   the ester resulting from the esterification reaction of hydrogenated castor oil with isostearic acid in a ratio of 1 to 3 (1/3) or hydrogenated castor oil triisostearate,
   and mixtures thereof.

Among the pasty compounds of plant origin that may be used, non-limiting mention may be made of an oxyethylenated (5 EO) oxypropylenated (5 PO) mixture of soybean sterols and of pentaerythritol, sold under the reference Lanolide® by the company Vevy.

The at least one additional pasty compound can be present in an amount ranging from 1% to 99%, such as from 1% to 60%, for instance from 2% to 30% and 5% to 15% by weight, relative to the weight of the composition.

Hydrocarbon-Based Dispersant

The composition as disclosed herein can further comprise at least one hydrocarbon-based dispersant.

As used herein, the term “hydrocarbon-based dispersant” is understood to mean a compound comprising carbon and hydrogen atoms and at least one functional group chosen from hydroxyl, ester, ether (ethylene oxide), carboxylic and amide functional groups.

The at least one hydrocarbon-based dispersant of the present disclosure contains no fluorine atoms. The compound bears at least one functional group that has a strong affinity for the surface of the particles to be dispersed.

For example, the at least one hydrocarbon-based dispersant in the composition according to the present disclosure can be fluid at room temperature (25° C.), such as a liquid and/or can have a refractive index ≧1.45 at 20° C. (the refractive index being measured by refractometry).

The hydrocarbon-based dispersant can have the solubility parameters δd and δa according to the Hansen solubility space that satisfy the following conditions:
16.2≦δ_{d}≦20 (J/cm³)^{1/2}, for example, 16.3≦δ_{d}≦19 (J/cm³)^{1/2}, such as 16.9≦δ_{d}≦18 (J/cm³)^{1/2}
and
9.1≦δₐ≦20 (J/cm³)^{1/2}, for example, 10≦δₐ≦18.1 (J/cm³)^{1/2}, such as 12≦δₐ≦14.5 (J/cm³)^{1/2}.

The definition of the solubility parameters according to Hansen is well known to those skilled in the art and is described for instance, in the article by C. M. Hansen: “The three dimensional solubility parameters,” J. Paint Technol. 39, 105 (1967). The parameters are also described in Japanese Patent No. JP-A-08-109 121 from KAO and the document from D. W. van Krevelen “Properties of polymers” (1990), p. 190.

According to the Hansen parameters:

δ_{d} is the London dispersion forces derived from the formation of dipoles induced during molecular impacts;

δₚ is the Debye interaction forces between permanent dipoles;

δₕ is the specific interaction forces (such as hydrogen bonding, acicdbase, donor/acceptor, etc.).

The parameters δ_{d}, δₚ and δₕ are generally expressed in (J/cm³)^{1/2}. They are determined at room temperature (25° C.) and, for example, according to the calculation method indicated above in Japanese Patent No. JP-A-08-109 121.

In the composition according to the present disclosure, any fluid, and for instance, liquid, hydrocarbon-based dispersant or a mixture of fluid hydrocarbon-based dispersants that satisfies the above relationships may be used. In this case, the solubility parameters of the mixture are determined from those of the fluid hydrocarbon-based dispersants taken separately, according to the following relationships:

$${δ}_{Dmixt} = {\sum }_{i} xi {δ}_{Di} ; {δ}_{pmixt} = {\sum }_{i} xi {δ}_{pi} and {δ}_{hmixt} = {\sum }_{i} xi {δ}_{hi}$$

wherein xi is the volume fraction of the fluid hydrocarbon-based dispersant (i) in the mixture.

It is within the capacity of a person skilled in the art to determine the amounts of each fluid hydrocarbon-based dispersant to obtain a mixture of fluid hydrocarbon-based dispersants that satisfies the above relationships.

For example, the hydrocarbon-based dispersant can have a chemical structure comprising at least one polar group chosen from —COOH; —OH; ethylene oxide —(O—CH₂—CH₂—); propylene oxide 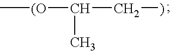
—PO₄; NHR; NR₁R₂ with R₁ and R₂ possibly forming a ring and wherein R₁ and R₂ are chosen from linear and branched C₁ to C₂₀ alkyl and alkoxy radicals, or 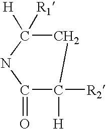
wherein R₁′ and R₂′ can be chosen from hydrogen atoms and linear and branched C₁ to C₂₀ alkyl and alkoxy chains.

The at least one hydrocarbon-based dispersant according to the present disclosure can be chosen from:
ether-modified fatty alcohols, for instance, the products of addition of ethylene oxide and/or of propylene oxide with i) a linear or branched fatty alcohol or with ii) an alkylphenol,
esters resulting from the reaction of at least one fatty acid with at least one product of addition of ethylene oxide and of glycerol or with at least one product of addition of ethylene oxide and of polyglycerol,
esters resulting from the reaction of glycerol or of polyglycerol with at least one product of addition of ethylene oxide and of a saturated or unsaturated fatty acid,
partial esters resulting from the reaction of at least one linear or branched, saturated or unsaturated fatty acid, of ricinoleic acid or of 12-hydroxystearic acid, with at least one polyol such as glycerol, polyglycerol, pentaerythritol, saccharide alcohols such as sorbitol, and for example, polyglycerol esters,
esters resulting from the reaction of sorbitan with at least one linear or branched, saturated or unsaturated fatty acid,
ether-modified sorbitan esters, for instance the esters resulting iii) from the reaction of sorbitan with at least one product of addition of ethylene oxide and of a saturated or unsaturated fatty acid, or iv) of the reaction of at least one saturated or unsaturated fatty acid with at least one product of addition of ethylene oxide and of sorbitan,
products of addition of ethylene oxide with castor oil and/or hydrogenated castor oil,
trialkyl phosphates and alkyl mono-, di- and triphosphates, and mixtures thereof, these compounds satisfying the solubility parameters defined above.

As used herein, the term “ester” is understood to mean a monoester, a diester, a triester and, more generally, a polyester.

For example, the at least one hydrocarbon-based dispersant can be chosen from monoesters, diesters and esters resulting from a partial esterification, i.e. the final ester comprises at least one free —OH functional group.

For instance, the at least one hydrocarbon-based dispersant can be chosen from:
the products of addition of 2 mol to 30 mol of ethylene oxide and/or of 0 mol to 5 mol of propylene oxide with i) a linear or branched C₈ to C₄₀ and for instance, C₈ to C₂₂ fatty alcohol or with ii) an alkylphenol,
esters resulting from the reaction of at least one C₈ to C₄₀ such as C₈ to C₂₂ fatty acid with at least one product of addition of 1 mol to 30 mol of ethylene oxide and of glycerol or with at least one product of addition of 1 mol to 30 mol of ethylene oxide and of polyglycerol,
esters resulting from the reaction of glycerol or of polyglycerol with at least one product of addition of 2 mol to 30 mol of ethylene oxide and of a saturated or unsaturated C₈ to C₄₀ such as a C₈ to C₂₂ fatty acid,
partial esters resulting from the reaction of at least one linear or branched, saturated or unsaturated C₈ to C₄₀, such as C₈ to C₂₂, fatty acid, of ricinoleic acid or of 12-hydroxystearic acid with glycerol, polyglycerol, pentaerythritol or sorbitol,
esters resulting from the reaction of sorbitan with at least one linear or branched, saturated or unsaturated C₈ to C₄₀, such as C₈ to C₂₂, fatty acid,
esters resulting iii) from the reaction of sorbitan with at least one product of addition of 2 mol to 30 mol of ethylene oxide and of a saturated or unsaturated C₈ to C₄₀, such as C₈ to C₂₂, fatty acid or iv) from the reaction of at least one saturated or unsaturated C₈ to C₄₀, such as C₈ to C₂₂, fatty acid with at least one product of addition of 2 mol to 30 mol of ethylene oxide and of sorbitan,
products of addition of 2 mol to 60 mol of ethylene oxide with castor oil and/or hydrogenated castor oil,
trialkyl phosphates and alkyl mono-, di- and triphosphates,
and mixtures thereof.

The at least one hydrocarbon-based dispersant can be chosen from, for example:

myristyl alcohol oxyethylenated with 15 ethylene oxide (or EO) groups (δ_{d}=17.33 (J/cm³)^{1/2} and δₐ=9.28 (J/cm³)^{1/2}), polyglyceryl-2 monoisostearate oxyethylenated with 5 EO (δ_{d}=17.34 (J/cm³)^{1/2} and δₐ=12.22 (J/cm³)^{1/2}), polyglyceryl-3 diisostearate (δ_{d}=16.96 (J/cm³)^{1/2} and δₐ=10.4 (J/cm³)^{1/2}), glyceryl monoisostearate (δ_{d}=16.32 (J/cm³)^{1/2} and δₐ=11.01 (J/cm³)^{1/2}), polyglyceryl-2 monoisostearate (δ_{d}=17.03 (J/cm³)^{1/2} and δₐ=13.25 (J/cm³)^{1/2}), polyglyceryl-3 isostearate (δ_{d}=17.38 (J/cm³)^{1/2} and δₐ=14.48 (J/cm³)^{1/2}), polyglyceryl-4 isostearate (δ_{d}=17.57 (J/cm³)^{1/2} and δₐ=15.37 (J/cm³)^{1/2}), polyglyceryl-6 monoisostearate (δ_{d}=17.86 (J/cm³)^{1/2} and δₐ=16.61 (J/cm³)^{1/2}), polyglyceryl-10 monoisostearate (δ_{d}=18.22 (J/cm³)^{1/2} and δₐ=18.41 (J/cm³)^{1/2}), polyglyceryl-2 monooleate (δ_{d}=17.14 (J/cm³)^{1/2} and δₐ=13.39 (J/cm³)^{1/2}), sorbitan isostearate (δ_{d}=17.33 (J/cm³)^{1/2} and δₐ=13.56 (J/cm³)^{1/2}), sorbitan monooleate (δ_{d}=17.32 (J/cm³)^{1/2} and δₐ=13.66 (J/cm³)^{1/2}), sorbitan monooleate oxyethylenated with 5 EO ((δ_{d}=17.56 (J/cm³)^{1/2} and δₐ=12.47 (J/cm³)^{1/2}), and mixtures thereof.

For further example, the at least one hydrocarbon-based dispersant can be chosen from partial esters of polyglycerol and of isostearic acid, partial esters of polyglycerol and of oleic acid and partial esters of sorbitan and of oleic acid, and mixtures thereof.

Among the hydrocarbon-based dispersants that may be used in the composition according to the present disclosure, non-limiting mention may be made of polyglyceryl-2 monoisostearate such as Salacos 41 manufactured or sold by the company Nisshin Oil Mills, polyglyceryl-3 diisostearate such as Lameform TGI manufactured or sold by the company Cognis, polyglyceryl-2 monooleate such as Rylo PG 29 manufactured or sold by the company Danisco Ingredients, or sorbitan monooleate such as Span 80 manufactured or sold by the company Uniqema, and mixtures thereof.

The at least one hydrocarbon-based dispersant can be present in an amount ranging from 0.5% to 40% by weight, for instance, from 3% to 20% by weight, such as from 5% to 15% by weight, relative to the total weight of the composition.

Dyestuff

The composition of the present disclosure may also comprise, for example, at least one dyestuff, which may be chosen from for instance, dyes that are soluble or dispersible in the composition, pigments and nacres, and mixtures thereof. In one embodiment of the present disclosure, the at least one dyestuff is a liposoluble dye; in another embodiment, the at least one dyestuff is chosen from water-soluble dyes. The at least one dyestuff can be present in an amount ranging from 0.001% to 98% by weight, for instance from 0.5% to 85% by weight, such as from 1% to 60% by weight, relative to the total weight of the composition.

For a composition in the form of a paste or a cast product such as lipsticks or body makeup products, the at least one dyestuff can be present in an amount ranging from 0.5% to 50%, for instance from 2% to 40%, such as from 5% to 30% by weight, relative to the total weight of the composition.

Among the liposoluble dyes that may be used, for example, non-limiting mention may be made of Sudan Red, D & C Red 17, D & C Green 6, β-carotene, soybean oil, Sudan Brown, D & C Yellow 11, D & C Violet 2, D & C Orange 5, quinoline yellow and annatto. The at least one liposoluble dye can be present in an amount ranging from 0% to 20%, for instance from 0.1% to 6% by weight, relative to the weight of the composition. Among the water-soluble dyes that may be used, non-limiting mention may be made of beetroot juice or methylene blue. The at least one water-soluble dye, when present, can be present in an amount ranging from 0.1 % to 6% by weight, relative to the total weight of the composition.

The composition of the present disclosure may also comprise, for example, a particulate phase, which can be colored for instance, and which, when present, may be present in an amount ranging from 0.001% to 50% by weight, for instance from 0.01% to 40% by weight, such as from 0.05% to 30% by weight, relative to the total weight of the composition, and which may also comprise at least one pigment and/or nacre and/or filler usually used in cosmetic compositions.

As used herein, the term “pigments” is understood to mean white or colored, mineral or organic particles, which are insoluble in the liquid fatty phase, and which are intended to color and/or opacify the composition. As used herein, the term “fillers” is understood to mean colorless or white, mineral or synthetic, lamellar or non-lamellar particles. As used herein, the term “nacres” is understood to meaning iridescent particles, produced for example by certain molluscs in their shell or alternatively synthesized. These fillers and nacres can be used, for instance, to modify the texture of the composition.

The at least one pigment, when present, may be present in the composition as disclosed herein, in an amount ranging from 0.05% to 30% byweight, for example, in an amount ranging from 2% to 20% by weight, relative to the weight of the final composition. Among the mineral pigments that may be used in the present disclosure, non-limiting mention may be made of titanium oxide, zirconium oxide, cerium oxide, zinc oxide, iron oxide, chromium oxide and ferric blue. Among the organic pigments that may be used in the present disclosure, non-limiting mention may be made of carbon black and bariurn, strontium, calcium (D & C Red No. 7) and aluminium lakes.

The at least one nacre, when present, may be present in the composition in an amount ranging from 0.001% to 20% by weight, such as in an amount ranging from 1% to 15% by weight, relative to the total weight of the composition. Among the nacres that may be used in the present disclosure, non-limiting mention may be made of mica coated with titanium oxide, with iron oxide, with natural pigment or with bismuth oxychloride, such as colored titanium mica.

The composition may also comprise at least one goniochromatic pigment, for example multilayer interference pigments, and/or reflective pigments. These two types of pigment are described in French Patent Application No. FR 02/09246, now published as FR 2 842 417, the content of which is incorporated herein by reference.

Fillers

The composition as disclosed herein may further comprise at least one filler. The at least one filler may be present, when present, in an amount ranging from 0.001% to 35% by weight, such as from 0.5% to 15% by weight, relative to the total weight of the composition.

Among the fillers that may be used, non-limiting mention may be made of
talc, mica, kaolin, starch,
Nylon® (for instance Organsol) powders
polyethylene powders,
polytetrafluoroethylene (Teflon®) powders,
boron nitride,
copolymer microspheres such as Expancel® (Nobel Industrie),
Polytrap® 603 (Dow Corning),
Polypore® L 200 (Chemdal Corporation),
silicone resin microbeads (for example Tospearl® from Toshiba),
silica-based fillers, for instance Aerosil 200 and Aerosil 300; Sunsphere L-31 and Sunsphere H-31 sold by Asahi Glass; Chemicelen sold by Asahi Chemical; composites of silica and of titanium dioxide, for instance the TSG series sold by Nippon Sheet Glass
polyurethane powders, for instance powders of crosslinked polyurethane comprising a copolymer, the copolymer comprising trimethylol hexyllactone. For example, it is a copolymer of hexamethylene diisocyanate/trimethylol hexyllactbne. Such particles are commercially available, for example under the name Plastic Powder D-400® or Plastic Powder D-800® from the company Toshiki.

The at least one filler may be, for example, a filler with a mean particle size of less than or equal 100 μm, for instance, ranging from 1 μm and 50 μm, for example ranging from 4 μm to 20 μm.

The at least one filler may be of any form, such as spherical or in the form of platelets.

Wax

The composition may also comprise at least one wax. As used herein, the term “wax” is understood to mean a lipophilic fatty compound that is solid at room temperature (25° C.), which undergoes a reversible solid/liquid change of state, which has a melting point of greater than 30° C., which may be up to 200° C., and a hardness of greater than 0.5 MPa, and which has an anisotropic crystal organization in the solid state. By bringing the wax to its melting point, it is possible to make it miscible with oils and to form a microscopically homogeneous mixture, wherein upon returning the temperature of the mixture to room temperature, recrystallization of the wax in the oils of the mixture is obtained.

The waxes that may be used in the present disclosure are compounds that are solid at room temperature, intended to structure the composition, for instance in the form of a stick; they may be hydrocarbon-based waxes, fluoro waxes and/or silicone waxes and may be of plant, mineral, animal and/or synthetic origin. For example, in one embodiment of the present disclosure, the waxes may have a melting point of greater than 40° C., such as greater than 45° C.

Among the waxes that may be used in the present disclosure, non-limiting mention may be made of those generally used in cosmetics: they can be of natural origin, for instance beeswax, carnauba wax, candelilla wax, ouricoury wax, Japan wax, cork fibre wax, sugarcane wax, rice wax, montan wax, paraffin, lignite wax or microcrystalline wax, ceresin, ozokerite and hydrogenated oils, for instance jojoba oil; synthetic waxes, for instance the polyethylene waxes derived from the polymerization or copolymerization of ethylene and Fischer-Tropsch waxes, or alternatively fatty acid esters, for instance octacosanyl stearate, glycerides that are solid at 40° C. and for instance at 45° C., silicone waxes, for instance alkyl- or alkoxydimethicones comprising an alkyl or alkoxy chain of 10 to 45 carbon atoms, poly(di)methylsiloxane esters that are solid at 40° C. and whose ester chain comprises at least 10 carbon atoms, and mixtures thereof.

The composition according to the present disclosure in one embodiment comprises polyethylene wax with a weight-average molecular mass ranging from 300 to 700, such as 500 g/mol.

The at least one wax, when present, may be present in an amount ranging from 0.01% to 50% by weight, such as from 2% to 40%, and for example from 5% to 30% by weight, relative to the total weight of the composition.

Non-Volatile Oil

The composition may also comprise at least one non-volatile oil. The at least one non-volatile oil may be chosen from:
hydrocarbon-based oils of animal origin such as perhydrosqualene;
hydrocarbon-based plant oils such as liquid triglycerides of fatty acids comprising from 4 to 10 carbon atoms, for instance heptanoic or octanoic acid triglycerides, or jojoba oil;
linear or branched hydrocarbons of mineral or synthetic origin, such as liquid paraffins and derivatives thereof, and petroleum jelly;
fatty alcohols comprising from 12 to 26 carbon atoms, for instance octyldodecanol, 2-butyloctanol, 2-hexyldecanol, 2-undecylpentadecanol and oleyl alcohol;
fluoro oils that are optionally partially hydrocarbon-based and/or silicone based;
silicone oils, for instance volatile or non-volatile, linear or cyclic polydimethylsiloxanes (PDMSs); polydimethylsiloxanes comprising alkyl, alkoxy or phenyl groups, which are pendant or at the end of a silicone chain, these groups comprising from 2 to 24 carbon atoms; phenylsilicones, for instance phenyl trimethicones (such as the phenyl trimethicone sold under the trade name DC556 by Dow Corning), phenyl dimethicones, phenyltrimethylsiloxydiphenylsiloxanes, diphenyl dimethicones, diphenylmethyidiphenyltrisiloxanes and 2-phenylethyl trimethylsiloxysilicates;
fatty acids comprising from 12 to 26 carbon atoms, for instance oleic acid
non-volatile esters comprising less than 40 carbon atoms
esters and polyesters of diol dimer, such as esters of diol dimer and of fatty acid, and esters of diol dimer and of diacid.

The esters of diol dimer and of monocarboxylic acid may be obtained from a monocarboxylic acid comprising from 4 to 34 carbon atoms, such as from 10 to 32 carbon atoms, which acids are linear or branched, and saturated or unsaturated.

As illustrative, non-limiting examples of monocarboxylic acids that are suitable in the invention, mention may be made of, for example, fatty acids.

The esters of diol dimer and of dicarboxylic acid may be obtained from a diacid dimer derived for instance from the dimerization of a C₈ to C₃₄, such as C₁₂ to C₂₂, for example C₁₆ to C₂₀ and such as C₁₈ unsaturated fatty acids.

According to one embodiment of the present disclosure, it is the diacid dimer from which the diol dimer to be esterified is also derived.

The esters of diol dimer can be obtained from a diol dimer produced by catalytic hydrogenation of a diacid dimer as described above, for example hydrogenated dilinoleic diacid.

As illustrations of esters of diol dimer, non-limiting mention may be made for example, of the esters of dilinoleic diacids and of dilinoleyl diol dimers sold by the company Nippon Fine Chemical under the trade name Lusplan DD-DA5® and DD-DA7®.
and mixtures thereof.

Non-volatile oils of plant or synthetic origin, for example, can be used.

The at least one non-volatile oil, when present, may be present in an amount ranging from 0.001% to 90% by weight, for instance, from 0.05% to 60%, such as from 1% to 35% by weight, relative to the total weight of the composition.

Additives

The composition of the present disclosure may also comprise at least one additional additive chosen from those used in the field under consideration, such as water, antioxidants, preserving agents, neutralizers, lipophilic gelling agents or non-aqueous liquid compounds, dispersants and cosmetic active agents. The at least one additive, when present, may be present in the composition in an amount ranging from 0.0005% to 20% by weight, such as from 0.001% to 10% by weight, relative to the total weight of the composition, with the exception of water, which may be present in an amount ranging from 0% to 70% by weight, for example from 1% to 50% by weight, such as from 1% to 10% by weight, relative to the total weight of the composition.

Among the cosmetic active agents that may be used in the present disclosure, non-limiting mention may be made of vitamins A, E, C, B₃ and F, provitamins, for instance D-panthenol, glycerol, calmative active agents, for instance α-bisabolol, aloe vera, allantoin, plant extracts or essential oils, protecting agents or restructuring agents, for instance ceramides, “refreshing” active agents, for instance menthol and its derivatives, emollients (cocoa butter, dimethicone), moisturizers (arginine PCA), anti-wrinkle active agents, essential fatty acids and sunscreens, and mixtures thereof.

Needless to say, a person skilled in the art will take care to select the optional additional additives and/or the amount thereof such that the beneficial properties of the composition according to the present disclosure are not, or are not substantially, adversely affected by the envisaged addition.

Galenical Forms

The compositions according to the present disclosure have numerous applications and concern all colored or uncolored cosmetic products, such as lipsticks.

The composition of the present disclosure may be in the form of a composition that is solid, compacted or cast, for instance in stick or dish form, pasty or liquid. It is can be, for example, in solid form, i.e. in hard form (which does not flow under its own weight) such as cast or compacted, for example in stick or dish form.

It maybe in the form of a paste, a solid or a cream. It may be an oil-in-water or water-in-oil emulsion, a solid or soft anhydrous gel, or alternatively in the form of a free or compacted powder and even in two-phase form. In one embodiment of the present disclosure, the composition is in the form of a composition with an oily and especially anhydrous continuous phase; in this case, it may comprise an aqueous phase in an amount of less than 5%.

The composition according to the present disclosure may be in the form of a colored or uncolored skincare composition, in the form of an antisun or makeup-removing composition or alternatively in the form of a hygiene composition. If it comprises cosmetic active agents, it may then be used as a care base or a non-therapeutic treatment base for the skin such as the hands or the face or for the lips (such as lip balms, for protecting the lips from cold and/or sunlight and/or wind) or an artificial tanning product for the skin.

The composition of the present disclosure may also be in the form of a colored makeup product for the skin, for example, for the face, for instance a blusher, a foundation, a makeup rouge or an eye-shadow, a makeup product for the body, for instance a semi-permanent tattoo product, or a makeup product for the lips, for instance a lipstick or a lip gloss, optionally having care properties or non-therapeutic treatment properties, a makeup product for the integuments, for instance a nail varnish, a mascara or an eyeliner, or a hair dye product or haircare product.

In one embodiment, the composition according to the present disclosure is in the form of a lipstick or a lip gloss.

Needless to say, the composition of the present disclosure should be physiologically acceptable (such as cosmetically acceptable), i.e., non-toxic and able to be applied to human skin, integuments or lips.

As used herein, the term “cosmetically acceptable” is understood to mean having a pleasant taste, feel, appearance and/or odor, applicable several days for several months.

The composition according to the present disclosure may be manufactured by the known processes generally used in cosmetics.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The examples that follow are intended to illustrate the present disclosure in a non-limiting manner. The amounts are given as percentages by mass.

### EXAMPLES

### Example 1

### Lipstick in Stick Form

| | Weight |
|---|---|
| Starting Materials | Percentage |
| Polyvinyl laurate | 6.5 |
| Polyester of dilinoleic acid dimer and of hydrogenated | 9.2 |
| castor oil (Risocast DA-L ® from the company Kokyu | |
| Alcohol Kogyo) | |
| PVP/eicosene | 7.4 |
| Microcrystalline wax | 3.9 |
| Ozokerite wax | 2.7 |
| N-Lauroyl-L-lysine | 2.0 |
| Kaolin | 2.0 |
| Preserving agents | 0.2 |
| Hydrogenated polyisobutene | 17.0 |
| Phenyl trimethicone sold or manufactured by the company | 17.0 |
| Dow Corning under the reference DC 556 | |
| Sorbitan oleate | 2.8 |
| Span 80 V from ICI-Uniqema | |
| Pigments | qs |
| Fillers | 4.0 |

### Example 2

### Lipstick in Stick Form

| | Weight |
|---|---|
| Starting Materials | Percentage |
| Ozokerite wax | 2 |
| Brown iron oxide (CI: 77491) | 4.0 |
| Microcrystalline wax | 3 |
| Sorbitan oleate | 7.2 |
| Span 80 V from ICI-Uniqema | |
| Polyvinyl laurate | 9.7 |
| Vinyl acetate/allyl stearate copolymer (65/35) | 1 |
| Mexomer PQ (P 2724 from Chimex) | |
| Glyceryl mono/di/tribehenate | 3 |
| Compritol 888 ATO from Gattefosse | |
| Phenyltrimethylsiloxytrisiloxane (viscosity: 20 cSt - DC | 2.8 |
| 556) | |
| Ethylene glycol dimethacrylate/lauryl methacrylate | 1 |
| copolymer | |
| Polytrap 603 Ultra-Lite Sorption Polymer Powder from | |
| Lambert-Riviere | |
| Isohexadecane | 17 |
| Brilliant blue FCF aluminium lake on alumina (12/88) (CI: | 0.1 |
| 42090: 2 + 77002) | |
| Calcium salt of Lithol Red B | 0.1 |
| Diisostearyl malate | 14.4 |
| Poly phenyltrimethylsiloxy dimethylsiloxane (viscosity: | 7.6 |
| 1000 cSt - Belsil 100) | |
| Alumina/treated rutile titanium | 2.2 |
| oxide/silica/trimethylolpropane (CI: 77891) | |
| Dilinoleyl diol dimers/dilinoleic dimers copolymer | 15 |
| Lusplan DD-DA7 from Nippon Fine Chemical | |
| Polyester of dilinoleic acid dimer and of hydrogenated | 9.3 |
| castor oil (Risocast DA-L ® from the company Kokyu | |
| Alcohol Kogyo) | |
| Preserving agent | qs |
| Total | 100 |

What is claimed is:

1. A cosmetic composition, comprising, in a cosmetically acceptable medium,
i) at least one polyester derived from the esterification, with a polycarboxylic acid, of an aliphatic hydroxycarboxylic acid ester comprising at least two hydroxyl groups and
ii) at least one volatile oil.

2. The cosmetic composition according to claim 1, wherein the polycarboxylic acid is an aliphatic polycarboxylic acid.

3. The cosmetic composition according to claim 2, wherein the polycarboxylic acid is an aliphatic dicarboxylic acid.

4. The cosmetic composition according to claim 2, wherein the polycarboxylic acid is a diacid dimer derived from at least one unsaturated fatty acid.

5. The cosmetic composition according to claim 4, wherein the dimer is chosen from unsaturated C₈ to C₃₄ fatty acid dimers.

6. The cosmetic composition according to claim 5, wherein the dimer is chosen from unsaturated C₁₂ to C₂₂ fatty acid dimers.

7. The cosmetic composition according to claim 6, wherein the dimer is chosen from unsaturated C₁₆ to C₂₀ fatty acid dimers.

8. The cosmetic composition according to claim 7, wherein the dimer is a C₈ fatty acid dimer.

9. The cosmetic composition according to claim 4, wherein the at least one unsaturated fatty acid is chosen from undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, and arachidonic acid.

10. The cosmetic composition according to claim 9, wherein the unsaturated fatty acid is linoleic acid and the dimer is dilinoleic acid.

11. The cosmetic composition according to claim 4, wherein the diacid dimer is saturated, and is derived from condensation of at least one unsaturated fatty acid optionally followed by a hydrogenation, to convert any double bonds into single bonds.

12. The cosmetic composition according to claim 1, wherein the aliphatic hydroxycarboxylic acid ester is chosen from:
a) partial or total esters of saturated linear monohydroxylated aliphatic monocarboxylic acids;
b) partial or total esters of unsaturated monohydroxylated aliphatic monocarboxylic acids;
c) partial or total esters of a C₂ to C₁₆ aliphatic polyol that has reacted with a monohydroxylated or polyhydroxylated aliphatic monocarboxylic or polycarboxylic acid;
and mixtures thereof.

13. The cosmetic composition according to claim 12, wherein the aliphatic hydroxycarboxylic acid ester is chosen from esters of C₂ to C₁₆ aliphatic polyols, wherein: the polyols have reacted with a hydroxylated aliphatic fatty acid with a saturated or unsaturated chain comprising at least 12 carbon atoms.

14. The cosmetic composition according to claim 12, wherein the aliphatic hydroxycarboxylic acid ester is chosen from saturated or unsaturated esters of C₂ to C₁₆ aliphatic polyols, wherein the polyols have reacted with ricinoleic acid.

15. The cosmetic composition according to claim 12, wherein the aliphatic hydroxycarboxylic acid ester is hydrogenated castor oil.

16. The cosmetic composition according to claim 1, wherein the at least one polyester has a liquid fraction and a solid fraction at 23° C., and a hardness ranging from 0.001 MPa to 0.5 MPa.

17. The cosmetic composition according to claim 16, wherein the at least one polyester has a liquid fraction and a solid fraction at 23° C., and a hardness ranging from 0.002 MPa to 0.4 MPa.

18. The cosmetic composition according to claim 1, wherein the at least one polyester is present in an amount ranging from 1% to 99% by weight, relative to the total weight of the composition.

19. The cosmetic composition according to claim 18, wherein the at least one polyester is present in an amount ranging from 1% to 75% by weight, relative to the total weight of the composition.

20. The cosmetic composition according to claim 19, wherein the at least one polyester is present in an amount ranging from 5% to 60% by weight, relative to the total weight of the composition.

21. The cosmetic composition according to claim 1, wherein the at least one volatile oil is chosen from
linear and cyclic silicones with a viscosity at room temperature of less than or equal to 8 cSt, these silicones optionally comprising alkyl or alkoxy groups comprising from 1 to 10 carbon atoms, and
volatile hydrocarbon-based oils comprising from 8 to 16 carbon atoms.

22. The cosmetic composition according to claim 21, wherein the linear and cyclic silicones comprise from 2 to 7 silicon atoms.

23. The cosmetic composition according to claim 21, wherein the linear and cyclic silicones are chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane.

24. The cosmetic composition according to claim 21, wherein the volatile hydrocarbon-based oils are chosen from branched C₈-C₁₆ alkanes and branched C₈-C₁₆ esters.

25. The cosmetic composition according to claim 24, wherein the branched C₈-C₁₆ alkanes are chosen from C₈-C₁₆ isoalkanes.

26. The cosmetic composition according to claim 1, wherein the at least one volatile oil is present in an amount ranging from 20% to 50% by weight, relative to the total weight of the composition.

27. The cosmetic composition according to claim 26, wherein the at least one volatile oil is present in an amount ranging from 30% to 40% by weight, relative to the total weight of the composition.

28. The cosmetic composition according to claim 16, wherein the at least one volatile oil is present in the liquid fatty phase in an amount ranging from 40% to 60% by weight, relative to the weight of the liquid fatty phase.

29. The cosmetic composition according to claim 28, wherein the at least one volatile oil is present in the liquid fatty phase in an amount ranging from 45% to 55% by weight, relative to the weight of the liquid fatty phase.

30. The cosmetic composition according to claim 1, wherein it is in the form of a product chosen from lipstick, lip gloss, makeup rouge, eyeshadow, mascara, eyeliner, nail varnish, artificial tanning product for the skin, hair coloring product or haircare product.

31. The cosmetic composition according to claim 1, further comprising at least one dyestuff.

32. The cosmetic composition according to claim 1, further comprising at least one additive chosen from water, waxes, pasty compounds, fillers, antioxidants, preserving agents, neutralizers, lipophilic gelling agents or non-aqueous liquid compounds, aqueous phase-gelling agents, dispersants, and cosmetic active agents.

33. The cosmetic composition according to claim 1, wherein it is in cast or compacted form.

34. The cosmetic composition according to claim 1, wherein it is in anhydrous form.

35. The cosmetic composition according to claim 30, wherein it is in the form of a lipstick or a lip gloss.

36. A cosmetic method for imparting at least one property chosen from gloss, staying-power and comfort properties, to a film of a cosmetic composition comprising introducing into the composition
i) at least one polyester derived from the esterification, with a polycarboxylic acid, of an aliphatic hydroxycarboxylic acid ester comprising at least two hydroxyl groups and
ii) at least one volatile oil,
wherein the at least one polyester and the at least one volatile oil are present in a combined amount effective to impart to the film at least one property chosen from gloss, staying power, and comfort properties.

37. A method for making a composition having at least one property chosen from gloss, staying-power and comfort properties, comprising combining
i) at least one polyester derived from the esterification, with a polycarboxylic acid, of an aliphatic hydroxycarboxylic acid ester comprising at least two hydroxyl groups and
ii) at least one volatile oil,
wherein the at least one polyester and the at least one volatile oil are present in a combined amount effective to impart to the composition at least one property chosen from gloss, staying power, and comfort properties.
